(19) United States
(12) Patent Application Publication
(10) Pub. No.: US 2014/0350640 A1
(43) Pub. Date: Nov. 27, 2014

(54) Implantable Medical Device and Tool Sensors

(71) Applicants: Jim Patrick, Roseville (AU); Claudiu Treaba, Jericho, NY (US); Matthijs Killian, Mechelen (BE); Ernst von Wallenberg, Muelheim (DE); Chris James, Toulouse (FR); Joerg Pesch, Mechelen (BE)

(72) Inventors: Jim Patrick, Roseville (AU); Claudiu Treaba, Jericho, NY (US); Matthijs Killian, Mechelen (BE); Ernst von Wallenberg, Muelheim (DE); Chris James, Toulouse (FR); Joerg Pesch, Mechelen (BE)

(21) Appl. No.: 14/284,546

(22) Filed: May 22, 2014

Related U.S. Application Data
(60) Provisional application No. 61/826,040, filed on May 22, 2013.

(51) Int. Cl. A61N 1/36

(52) U.S. Cl. CPC A61N 1/36032
USPC 607/57

(57) ABSTRACT

Implantable medical devices and instruments (tools) including one or more implantable sensors. In certain embodiments, a hearing prosthesis comprises an implantable stimulating assembly configured to be implanted in a recipient's cochlea. At least one implantable sensor is disposed in the implantable stimulating assembly and is configured to monitor an insertion attribute.

### CROSS-REFERENCE TO RELATED APPLICATIONS

This application claims priority to U.S. Provisional Application No. 61/826,040 entitled “IMPLANTABLE MEDICAL DEVICE AND TOOL SENSORS,” filed May 22, 2013, the content of which is hereby incorporated by reference herein.

### BACKGROUND

1. Field of the Invention

The present invention relates generally to implantable medical devices.

2. Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., auditory brain stimulators, bone conduction devices, mechanical stimulators, middle ear implants, cochlear implants, etc.), implantable pacemakers, defibrillators, functional electrical stimulation devices, and other implantable medical devices, have been successful in performing life saving and/or lifestyle enhancement functions for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional components perform diagnosis, prevention, monitoring, treatment or management of a disease or injury or symptom thereof, or to investigate, replace or modify the anatomy or of a physiological process.

### SUMMARY

In one aspect, a hearing prosthesis is provided. The hearing prosthesis comprises an implantable stimulating assembly configured to be implanted in a recipient's cochlea, and at least one implantable sensor disposed in the implantable stimulating assembly configured to monitor an insertion attribute.

In another aspect, a method for implanting a hearing prosthesis in a recipient is provided. The method comprises inserting an implantable stimulating assembly in a cochlea of the recipient, and during insertion of the implantable stimulating assembly, monitoring an insertion attribute with at least one implantable sensor disposed in the implantable stimulating assembly.

### BRIEF DESCRIPTION OF THE DRAWINGS

Embodiments of the present invention are described herein in conjunction with the accompanying drawings, in which:

FIG. 1 is a schematic diagram of a cochlear implant system having an implantable sensor in accordance with embodiments presented herein;

FIG. 2A is a side view of the implantable component of the cochlear implant system of FIG. 1;

FIG. 2B is a perspective view of a distal region of the stimulating assembly of the implantable component of FIG. 2A;

FIG. 3 is a perspective view of a tool in accordance with embodiments presented herein that includes an implantable sensor;

FIG. 4A is a simplified cross-sectional view of a stimulating assembly held straight by a stiffening element that includes an implantable sensor;

FIG. 4B is a simplified cross-sectional view of a stimulating assembly held straight by two stiffening elements that each include an implantable sensor;

FIG. 5 is a perspective view of a tool in accordance with embodiments presented herein that includes a sensor;

FIGS. 6A and 6B illustrate a recipient's cochlea in which a stimulating assembly having an implantable sensor may be inserted;

FIG. 6C is a schematic diagram illustrating an implantable sensor for detecting dislocation of a stimulating assembly in accordance with embodiments of the present invention;

FIG. 6D is a schematic diagram illustrating use of the implantable sensor of FIG. 6C;

FIGS. 7A-7C are schematic diagrams illustrating a stimulating assembly having electrical sensing electrodes in accordance with embodiments presented herein;

FIG. 8 is a schematic diagram illustrating a stimulating assembly having a low impedance electrode for recording afterpotentials in accordance with embodiments presented herein;

FIG. 9 is a schematic diagram illustrating use of an implantable sensor in accordance with embodiments presented herein;

FIG. 10 is a flowchart of a method in accordance with embodiments presented herein;

FIG. 11 is schematic diagram illustrating a bone conduction device having an implantable sensor in accordance with embodiments presented herein;

FIG. 12 is schematic diagram illustrating a bone conduction device having an implantable sensor in accordance with embodiments presented herein; and

FIG. 13 is schematic diagram illustrating a mechanical stimulating having an implantable sensor in accordance with embodiments presented herein.

### DETAILED DESCRIPTION

Embodiments presented herein are generally directed to implantable sensors used in conjunction with implantable medical devices and instruments (tools). There are many different types of implantable medical devices having a wide variety of corresponding implantable components that may be partially or fully implanted into a recipient. For example, implantable medical devices may include hearing prostheses (e.g., auditory brain stimulators, bone conduction devices, mechanical stimulators, middle ear implants, cochlear implant systems, etc.), implantable pacemakers, defibrillators, functional electrical stimulation devices, catheters, etc. There are also a large number of different types of tools that may be used in with these implantable medical devices. For example, different types of tools are used during implantation, explantation, etc. of an implantable medical device. Merely for ease of illustration, the implantable sensors will be primarily described herein in connection with a cochlear implant system (cochlear implant) and tools used during implantation of a cochlear implant. However, the implantable sensors described herein may be used with any of the above or other tools, devices, etc.

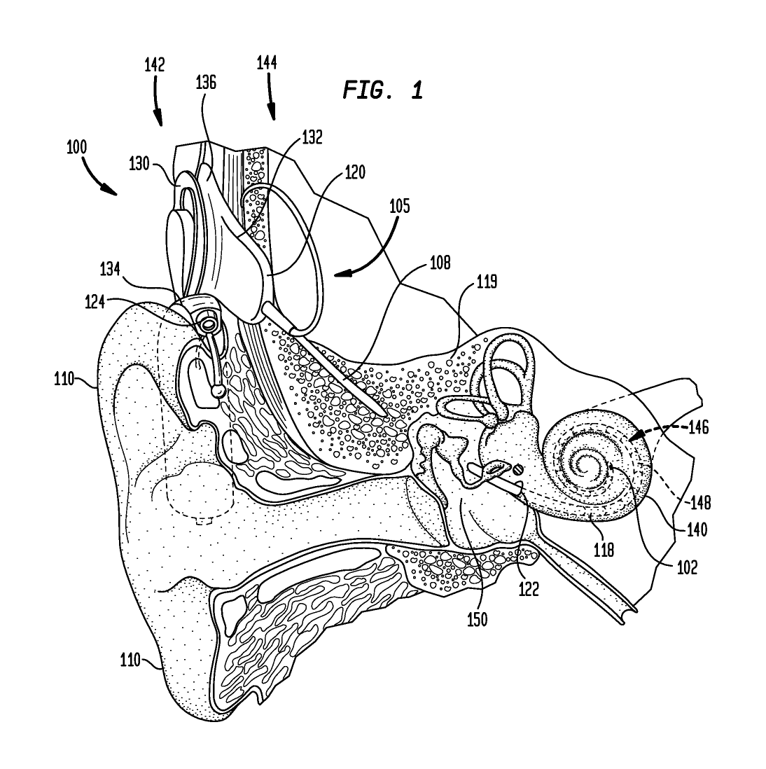

FIG. 1 is perspective view of an exemplary cochlear implant 100 that comprises an implantable sensor in accordance with embodiments presented herein. The cochlear implant 100 includes an external component 142 and an internal or implantable component 144. The external component 142 is directly or indirectly attached to the body of the recipient and typically comprises one or more sound input elements 124 (e.g., microphones, telecoils, etc.) for detecting sound, a sound processor 126, a power source (not shown), an external coil 130 and, generally, a magnet (not shown) fixed relative to the external coil 130. The sound processor 126 processes electrical signals generated by a sound input element 124 that is positioned, in the depicted embodiment, by auricle 110 of the recipient. The sound processor 126 provides the processed signals to external coil 130 via a cable (not shown).

The internal component 144 comprises an implant body 105, a lead region 108, and an elongate stimulating assembly 118. The implant body 105 comprises a stimulator unit 120, an internal coil 136, and an internal receiver/transceiver unit 132, sometimes referred to herein as transceiver unit 132. The transceiver unit 132 is connected to the internal coil 136 and, generally, a magnet (not shown) fixed relative to the internal coil 136. Internal transceiver unit 132 and stimulator unit 120 are sometimes collectively referred to herein as a stimulator/transceiver unit 120.

The magnets in the external component 142 and internal component 144 facilitate the operational alignment of the external coil 130 with the internal coil 136. The operational alignment of the coils enables the internal coil 136 to transmit/receive power and data to/from the external coil 130. More specifically, in certain examples, external coil 130 transmits electrical signals (e.g., power and stimulation data) to internal coil 136 via a radio frequency (RF) link. Internal coil 136 is typically a wire antenna coil comprised of multiple turns of electrically insulated single-strand or multi-strand platinum or gold wire. The electrical insulation of internal coil 136 is provided by a flexible silicone molding. In use, transceiver unit 132 may be positioned in a recess of the temporal bone of the recipient. Various other types of energy transfer, such as infrared (IR), electromagnetic, capacitive and inductive transfer, may be used to transfer the power and/or data from an external device to cochlear implant and FIG. 1 illustrates only one example arrangement.

Elongate stimulating assembly 118 is implanted in cochlea 140 and includes a contact array 146 comprising a plurality of stimulating contacts 148. Stimulating assembly 118 extends through cochleostomy 122 and has a proximal end connected to stimulator unit 120 via lead region 108 that extends through mastoid bone 119. In the embodiments of FIG. 1, an implantable sensor 102 is disposed at the distal end (tip) of the as stimulating assembly 118.

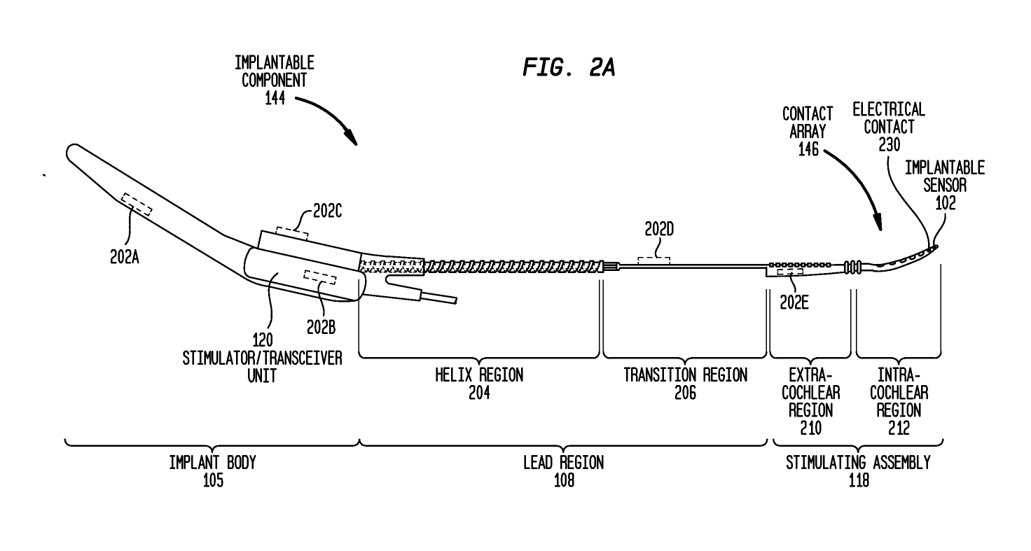

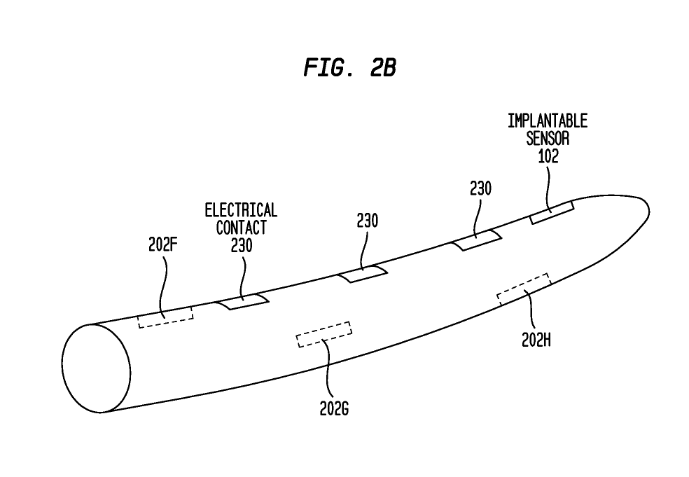

FIG. 2A is a simplified side view of implantable component 144 of FIG. 1, while FIG. 2B is a perspective view of the distal region of stimulating assembly 118. As shown, the stimulating assembly 118 comprises an extra-cochlear region 210 and an intracochlear region 212. Intra-cochlear region 212 is configured to be implanted in the recipient's cochlea and has disposed thereon a contact array 146. In the present example, contact array 146 comprises electrical stimulating contacts 230. In alternative embodiments, the contact array 146 may also or alternatively comprise optical stimulating contacts or combination of electrical stimulating contacts and optical stimulating contacts.

Implantable component 144 further comprises a lead region 108 coupling the stimulating assembly 118 to implant body 105 and, more particularly, stimulator/transceiver unit 120. Lead region 108 comprises a region 204 which is commonly referred to as a helix region, however, the required property is that the lead accommodate movement and is flexible, it does not need to be formed from wire wound helically. Lead region 108 also comprises a transition region 206 which connects helix region 204 to stimulating assembly 118. Electrical and/or optical stimulation signals generated by stimulator/receiver unit 120 are delivered to contact array 146 via lead region 108. Helix region 204 prevents lead region 108 and its connection to stimulator/receiver 120 and stimulating assembly 118 from being damaged or impaired due to movement of internal component 144 (or part of 144) which may occur, for example, during mastication.

As shown in FIGS. 2A and 2B, an implantable sensor 102 is disposed at the distal end (tip) of the stimulating assembly 118. In general, a sensor is a component configured to gather some type of information (e.g., by sensing/sampling/monitoring some parameters) and configured to convert the information into a signal. An “implantable” sensor is a sensor that has a configuration such that it will operate when it is inserted (e.g., temporarily or permanently implanted) into a recipient. As such, an “implantable sensor,” such as implantable sensor 102, is able to gather information and convert the information into signals while positioned in a recipient.

It is to be appreciated that distal tip location of implantable sensor 102 in FIG. 1 is merely illustrative and that implantable sensors may be positioned at various other locations in/on implantable component 144. FIGS. 2A and 2B illustrate a plurality of other potential implantable sensor locations using dashed boxes 202A to 202H. For example, dashed box 202A illustrates an implantable sensor positioned in or near the magnet in the stimulator/transceiver unit 120 that couples to the magnet of an external component (e.g., in the magnet pocket of the stimulator/transceiver unit 120). Other locations for an implantable sensor include, without limitation, other portions of the implant body (shown by dashed boxes 202B and 202C), the lead region 108 (shown by dashed box 202D), the extra-cochlear region 210 of stimulating assembly 118 (shown by dashed box 202E), or portions of the intra-cochlear region other than the distal tip (shown by dashed boxes 202F, 202G, and 202H). The location for an implantable sensor may depend, for example, on the type of sensor and/or the sensed parameter(s) (i.e., the type of information the sensor is designed to gather).

Implantable sensors in accordance with embodiments presented herein may be one of a number of different types of sensors configured to perform a variety of functions. For example, implantable sensors may be configured to sense various physical, biological, chemical, and/or electrical parameters. Additionally, implantable sensors may assist in placement of the stimulating assembly within the cochlea and/or assist with the avoidance of, confirmation of, or confirmation of the absence of, trauma.

In certain embodiments, an implantable sensor is configured to monitor an “insertion attribute.” As used herein, an insertion attribute refers to a characteristic or aspect of the stimulating assembly insertion process (i.e., the process during which a stimulating assembly is implanted in a recipient's cochlea). For example, hearing loss may occur when a stimulating assembly is inserted into a recipient's cochlea due to, at least in part, the speed of the stimulating assembly insertion. More specifically, when the stimulating assembly is inserted into the cochlea too quickly, the fluid pressure within the cochlea increases to a point that can damage residual hearing. This pressure increase may also be transferred into the vestibular system that can cause temporary or permanent damage to the vestibular system (e.g., damage the hair cells within the cochlea). As such, in one embodiment presented herein, the implantable sensor 102 positioned at the distal tip of the stimulating 118 may comprise a pressure sensor configured to measure the pressure in the perilymph during insertion (i.e., the perilymph fluid pressure is the insertion attribute monitored by the implantable sensor 102).

In one such embodiment, when the cochlear fluid pressure increases above a first pre-determined safety level (threshold value), an alert or feedback (e.g., audible, visual, haptic, etc.) is given to the surgeon. The surgeon can then modify their insertion (e.g., temporarily halting insertion of the stimulating assembly, slowing the insertion, etc.) so that a potentially harmful pressure increase is avoided. This may improve the residual hearing outcomes of the recipient. Additionally, a second pre-determined safety level (threshold level) could be set that indicates that pressure has increased to a level that damage is believed to have occurred. In other words, a first threshold may be set at or below a pressure level where damage is likely to occur, and a second threshold may be set at above the pressure level where damage is likely to occur (i.e., one threshold that is within safety, and a second threshold that indicates beyond safety). In certain examples, different feedback may be used to indicate that the fluid pressure within the cochlea has reached each of the first and second thresholds.

The pre-determined threshold(s) at which a surgeon is alerted may vary depending on, for example, the problem that is to be avoided (i.e., one threshold may be selected to avoid damage to the cochlea hair cells, while another threshold may be selected to avoid damage to the vestibular system). The same or different pressure sensors may be used to alert at the different thresholds. The pressure sensor may also be used in various manners after the implantation process.

As noted, pressure sensor 102 is positioned at the distal tip of the stimulating assembly 118. In an alternative embodiment, an additional sensor outside the cochlea may be used to measure the deflection of the oval window/stapes/ossicular movement and correlate this with increase in pressure within the cochlea. Although acoustic stimulation may create noise in such a measure, this noise may be eliminated through measurement of environmental sounds.

In certain embodiments, the pressure sensor is a micro silicone pressure sensor. In addition to measuring the fluid pressure with the cochlea, the pressure sensor could also be used as a sound input element for a cochlea implant (i.e., to detect movement of the cochlea fluid in response to received sound)

The cochlear fluid pressure is not the only insertion attribute that may be monitored by an implantable sensor during insertion of a stimulating assembly. In further embodiments, the implantable sensor 102 may be a speed sensor (accelerometer) configured to monitor the speed or movement of the stimulating assembly during insertion (i.e., the speed of insertion of the stimulating assembly is a monitored insertion attribute). Alternatively the pressure sensor could be used to calculate a speed or acceleration of insertion by utilizing the changes of pressure over time.

In other embodiments, a speed sensor or accelerometer may be positioned in/on the lead region 108 and/or the implant body 105. In normal operation, the lead region 108 and/or the implant body 105 may move along with the recipient's skull/tissue (as the recipient moves the lead region 108 and/or the implant body 105 and the bone/tissue experience corresponding movement. A speed sensor or accelerometer positioned in/on the lead region 108 and/or the implant body 105 may be configured to alert when movement of the lead region and/or the implant body relative to the recipient's skull or other tissue is detected (i.e., when movement of lead region 108 and/or the implant body 105 does not correspond with the movement, or lack of movement, of the bone/tissue).

The implantable sensor 102 may be a force sensor configured to determine if the stimulating assembly impacts or comes in contact with a wall of the cochlea during insertion, or even the extend of the wall contact (e.g., showing tip wedging, too deep insertion, etc.). As such, contact (impact) between the stimulating assembly and a wall of the cochlea is a monitored insertion attribute). In such embodiments, impacts over a certain threshold that occur during insertion of a stimulating assembly may trigger feedback to the surgeon.

Additionally, impacts over a certain threshold that occur after implantation (e.g., during cochlear implant use by a recipient) could be detected by a sensor in the implant body. These impacts may trigger feedback that is transmitted to the implanted stimulator unit, sound processor, or equipment accessible by the recipient, a clinician, caregiver, etc. In certain circumstances, the system may generate an audible alert to, for example, a caregiver upon detection of an impact, the system be configured to record/log the number of impacts for subsequent use, etc.

In another embodiment, the implantable sensor 102 may comprise a proximity sensor that is configured to detect when the stimulating assembly is close to a cochlear wall and/or provide an indication of the distance between the stimulating assembly and a cochlea wall (i.e., proximity of the stimulating assembly to a cochlea wall is a monitored insertion attribute). Such proximity sensors may assist with the final placement of the stimulating assembly in the cochlea. Proximity sensors may operate based on electrical, electrochemical or electro-neural properties (e.g., voltage, current, impedance, neural potential, light etc.) In certain embodiments, proximity sensors may provide an objective or defined measure of the proximity of the stimulating assembly to a cochlea structure. In other embodiments, the output from a proximity sensor may be used to produce a reconstructed image of the cochlea (or a portion thereof). Proximity sensors may also be positioned in/on the lead region 108 to, for example, assist in positioning of the lead region. In another embodiment, the proximity sensor may be positioned in/on the implant body.

In another embodiment, the implantable sensor 102 may be a sensor configured to track perilymph movement. In such embodiments, the implantable sensor 102 may generate an alert if no or little movement is detected over a predetermined period of time (e.g., 48 hours). Other implantable sensors may be used to track relative movement of other portions of the recipient's anatomy (e.g., a sensor in/on the lead region 108 to track movement of the recipient's middle ear bones).

In a further embodiment, the implantable sensor 102 may be any device configured to produce an image of, or assist in production of a reconstructed image of, the recipient's cochlea, mastoid cavity, etc., before, during, or after surgery. In such embodiments, the implantable sensor 102 may be an optical fiber, camera, ultrasound device, scanning device (e.g., x-ray element), etc., which is positioned in/on the stimulating assembly 118, the lead region 108, or the implant body 105.

Alternatively, implantable sensor 102 may comprise an orientation sensor. An orientation sensor may be configured to, for example, detect when the recipient is lying down. In response, the cochlear implant program may be automatically changed to, for example, only deliver softer sounds to the recipient (excluding alarms). Such an orientation sensor may be positioned in/on the stimulating assembly 118, the lead region 108, or the implant body 105.

In certain embodiments, the implantable sensor 102 may comprise an optical or electro-optical sensor. Such sensors are detectors that convert light, or a change in light, into an electronic signal. In certain embodiments, such a sensor may be positioned in/on a stimulating assembly that uses optical stimulation (e.g., detecting undesired light spread, measuring wavelength of reflected/absorbed optical signals from the cochlea nerve or hair cells, etc.). Such a sensor may be used in/on other stimulating assemblies that do not use optical stimulation, a lead region, or an implant body.

In another embodiment, the implantable sensor 102 may comprise a charge sensor that is configured to detect when an electrical contact such as the stimulating intra-cochlear electrical contacts or reference electrical contacts (inside or outside of the cochlea) are functioning improperly and/or have ceased functioning. As such, depending on the electrical contacts to be monitored, the charge sensor may be positioned in the intra-cochlear region 212 of the stimulating assembly 118, the extra-cochlear region 210 of the stimulating assembly 118, or the lead region 108. A charge sensor could also be in/on the lead region 108 or the implant body 105 to monitor other electrical components, such as the wires connecting the electrical contacts to the stimulator/transceiver unit 120, the internal coil 136 (FIG. 1), and/or other circuitry within the implant body 105.

In another embodiment, the implantable sensor 102 may be a temperature sensor rather than a pressure sensor. In certain circumstances, the temperature sensor is in/on the intra-cochlear region 212 and is configured to monitor the temperature within the cochlea. An increase in the temperature within the cochlea may indicate the presence of infection. As such, if the temperature rises above a predetermined safely level (threshold level), then feedback could be generated by the sensor 102. The feedback could initiate a corrective action such as, for example, the release of an anti-biotic drug.

A temperature sensor may also be positioned at other locations in/on the implantable component 144. In one embodiment, the temperature sensor may be positioned in/on the lead region 108 to, for example, monitor the temperature in the mastoid cavity. In another embodiment, the temperature sensor may be positioned in/on the implant body 105 to, for example, monitor the temperature of soft tissue and/or within the bone pocket (i.e., the cavity within the recipient's bone in which the implant body 105 is positioned). As such, if the temperature rises above a predetermined safely level (threshold level), then feedback could be generated by the sensor. The feedback could initiate an alert, such as a signal sent to the remote assistant (remote control) for the recipient to see a doctor or clinician for assistance.

In accordance with embodiments presented herein, implantable sensor 102 may comprise a biological sensor (biosensor). In general, a biological sensor is a device configured to monitor a biophysical or biochemical quantity or parameter into signals (e.g., electrical signals). Biological sensors may be disposed in/on the stimulating assembly 118, the lead region 108, and/or the implant body 105.

A biological sensor in accordance with embodiments presented herein may be configured to measure the presence of microorganisms, including bacteria, fungi, viruses, etc. Alternatively, biological sensors in accordance with embodiments presented herein may be configured to measure the presence of cells such as fibrotic growth on the stimulating contacts, bone cells, neuronal cells, etc. In still other embodiments, biological sensors presented herein may be configured to measure the presence of: proteins (proteins are possible indicators of reactions), DNA/RNA/siRNA, enzymatic activity, oxidative radicals, drugs (even of metabolized parts of the drug), ions (e.g., K+, Na+, etc.).

In certain embodiments, the implantable sensor 102 may comprise or operate with a biological marker (biomarker). A biological marker is an indicator of a biological state, or the past or present existence of a particular type of organism. For example, a biological marker may be used that is configured to activate in the presence of glucose. This activation could trigger an observable event that enables sensing of the biological state. Biological markers may be particularly useful as biosensors to measure the presence of insulin, the presence of DNA (the subsequent action could be a gene therapy, molecular process, etc.), presence of infection (the subsequent action could be antibiotic drug delivery), presence of inflammation (the subsequent action could be deliver of an anti-inflammatory drug delivery. In embodiments in which a drug is delivered, the implantable sensor 102 (or other sensor) could measure the effectiveness of the drug by continuing to detect the inflammation in order to determine if additional (or less) drugs are needed.

In accordance with embodiments presented herein, the biological markers may be attached to the surface of the stimulating assembly 118 (or other parts of the implantable component 144) prior to implantation. Once the stimulating assembly 118 is implanted, if a molecule of interest is detected in the cochlea, then the molecule will attach and react with the biomarkers. The reaction may then be detected by implantable sensor 102 via, for example, a change in a sensed electrical property such as impedance or current.

In certain embodiments, the implantable sensor 102 is configured to detect the build-up of biofilms or tissue on the stimulating assembly 118. In such embodiments, if build-up is detected on the stimulating assembly 118, an electrical charge may be sent across the stimulating assembly to reduce and/or deter growth (e.g., the electrical charge could be subthreshold or suprathreshold stimulation signals). Alternatively, a drug or other element may be released to coat the area so that a bacterium that may cause a negative reaction does not have any room to populate.

In further embodiments, implantable sensor 102 may be configured to detect ionic changes in the cochlea. For residual hearing, changes in the ionic level may change the hair cells, the spiral ganglion, the nerve fibers, etc. Detection of ionic changes may be useful for diagnostics and/or may be used to change stimulation parameters, release a drug, flag an intervention by a surgeon, etc. Further embodiments may make use of an osmotic sensor.

FIGS. 1, 2A, and 2B primarily illustrate an arrangement that includes only one implantable sensor 102. It is to be appreciated that alternative embodiments may use a plurality of sensors of the same or different type.

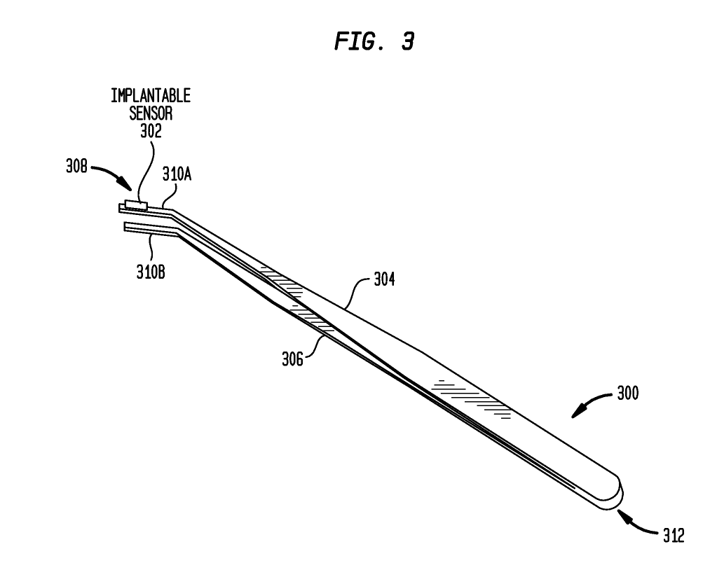

As noted above, in accordance with embodiments presented herein, sensors (e.g., implantable or nonimplantable sensors) may also be included in or on medical tools (instruments) used in conjunction with implantable medical devices (e.g., tools used during device implantation, explantation, etc.) FIGS. 3-5 illustrate various embodiments in which tools include sensors.

More specifically, FIG. 3 is a perspective view of an exemplary embodiment of a manual insertion tool for a stimulating assembly, such as stimulating assembly 118 of FIG. 1. As shown, the tool 300 has a body 302 which branches into two relatively flexibly movable arms 304 and 306. Arms 304 and 306 terminate at end 308 in respective tips 310A, 310B which are adapted to hold or capture a substantially tubular element, such as stimulating assembly 118, there between. Arms 304 and 306 are fixed together with respect to each other at one end 312, and are biased such that tips 310A and 310B of arms 304 and 306, when in a relaxed position, are positioned remote from each other at end 308. In this regard, tips 310A and 310B may be brought together by applying a compressive force on movable arms 304 and 306 to hold or capture an element between tips.

In accordance with embodiments presented herein, the tool 300 includes a sensor 302 positioned thereon. More specifically, as shown in FIG. 3, the sensor 302 is positioned on an outward facing surface of the tip 310A of arm 304 (i.e., on the surface facing away from the opposing tip 310B). The sensor 302 may be any of the sensors described above. For example, in one embodiment, the sensor 302 may be a force sensor. In another embodiment, the implantable sensor 302 may be a speed or movement sensor.

The location of sensor 302 shown is merely illustrative and other locations may be used in alternative embodiments. For example, in alternative embodiments the sensor 302 could be positioned on an inward facing surface of the tip 310A (i.e., on the surface facing towards from the opposing tip 310B), on the opposing tip 310B, or positioned on or in other portions of the arms 304 and 306. Additionally, although FIG. 3 illustrates the use of a single sensor 302, alternative embodiments may include a plurality of sensors disposed in or on tool 300.

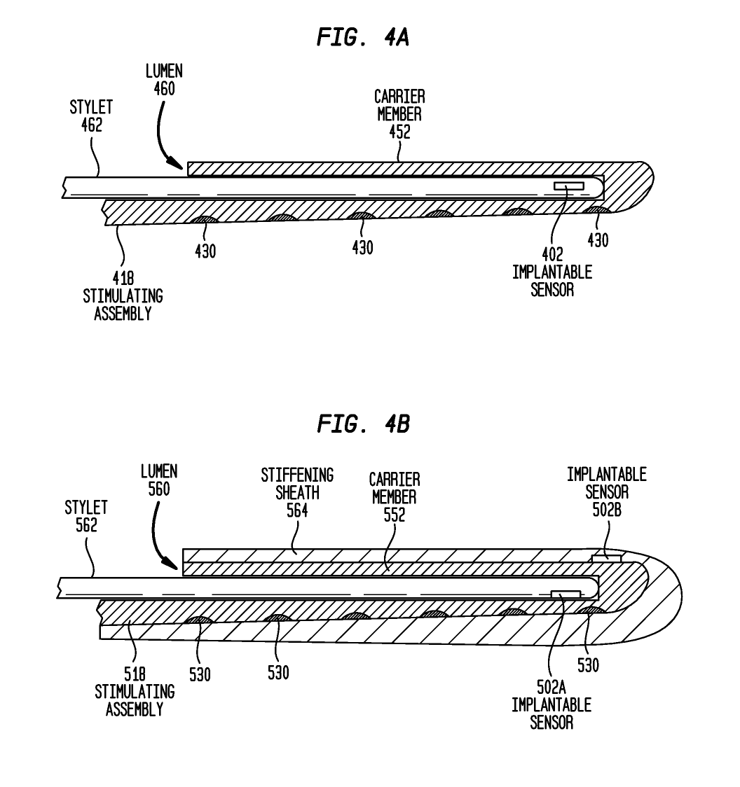

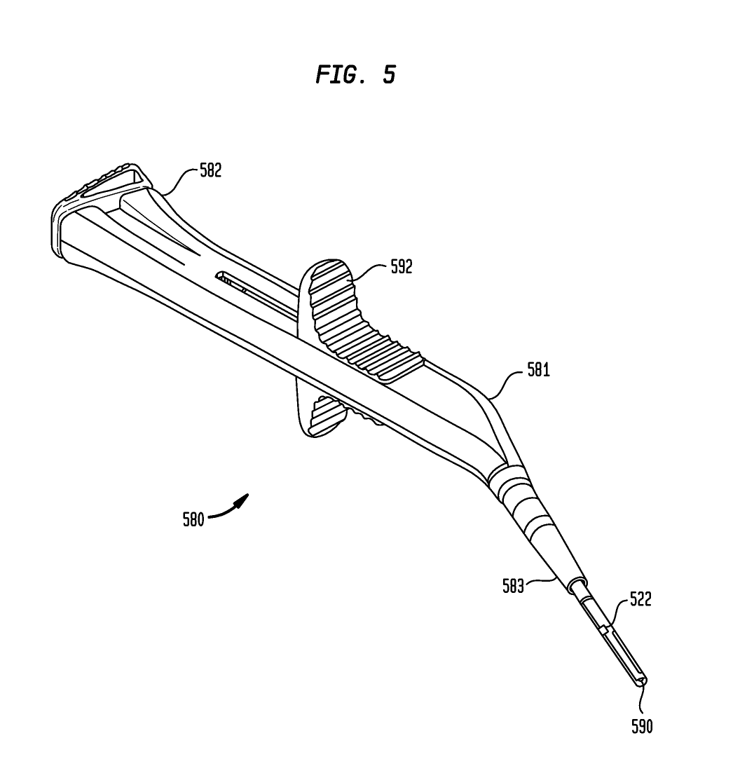

There are a variety of types of intra-cochlear stimulating assemblies including short, straight and perimodiolar. A perimodiolar stimulating assembly is configured to adopt a curved configuration during and or after implantation into the recipient's cochlea. To achieve this, in certain arrangements, a stimulating assembly is pre-curved to the same general curvature of a cochlea. In such examples, stimulating assembly is typically held straight by one or more tools, typically referred to as straightening elements, which are removed during implantation. These straightening elements may comprise, for example, stiffening stylets or stiffening sheathes. As shown in FIGS. 4A and 4B, these straightening elements may include one or more implantable sensors configured to, for example, assist in the implantation of a stimulating assembly into the cochlea.

More specifically, FIG. 4A is a simplified cross-sectional view of a portion of a stimulating assembly 418 comprising an elongate carrier member 452 having a plurality of electrical contacts 430 disposed therein. The stimulating assembly 418 is preformed from a resiliently flexible polymeric member that is formed into a pre-curved configuration suitable for insertion into a recipient's cochlea.

The carrier member 452 also has an elongate lumen 460 disposed therein. Positioned in the lumen 460 is a substantially straight platinum stylet 462. In the example of FIG. 4A, the stylet 462 has a stiffness that is sufficient to retain the carrier member 452 in a substantially straight configuration. During implantation of the stimulating assembly 418 into a recipient, a surgeon slow withdraws the stylet 462 from the lumen 460 so that the carrier member 452 returns to its pre-curved shape within the cochlea, thereby positioning the electrical contacts 430.

As shown in FIG. 4A, disposed in or on the stylet 462 is an implantable sensor 402. More specifically, as shown in FIG. 4A, the sensor 402 is positioned at a distal end of the stylet. The implantable sensor 402 may be any of the sensors described above. For example, in one embodiment, the implantable sensor 402 may be a force sensor. In another embodiment, the implantable sensor 402 may be a speed or movement sensor.

The location of implantable sensor 402 shown is merely illustrative and other locations may be used in alternative embodiments. Additionally, although FIG. 4A illustrates the use of a single implantable sensor 402, alternative embodiments may include a plurality of sensors disposed in or on stylet 462.

FIG. 4B is a simplified cross-sectional view of a portion of another stimulating assembly 518 comprising an elongate carrier member 552 having a plurality of electrical contacts 530 disposed therein. The stimulating assembly 518 is preformed from a resiliently flexible polymeric member that is formed into a pre-curved configuration suitable for insertion into a recipient's cochlea.

The carrier member 552 also has an elongate lumen 560 disposed therein. Positioned in the lumen 560 is a substantially straight platinum stylet 562. In the example of FIG. 4B, the stylet 562 has a stiffness that is insufficient to retain the carrier member 552 in a substantially straight configuration. As such, a second stiffening element, shown as stiffening sheath 564, is also provided. The stiffening sheath 564 is formed from a bioresorable material (i.e., a material adapted to dissolve or soften on exposure to cochlear fluids).

When the stimulating assembly 518 is inserted to a recipient's cochlea, the cochlear fluids commence to dissolve and/or soften the sheath 564. As the sheath 564 softens and/or dissolves, the carrier member 552 commences to return to its pre-curved shape (as the stiffness of the stylet 562 is insufficient to hold the carrier member straight). The provision of the stylet 562 in the lumen 560 does, however, prevent the carrier member 552 from fully adopting its pre-curved spirally curved configuration (i.e., the stylet 562 retains the carrier member 552 in an intermediate curved configuration).

As the carrier member 552 curls, the surgeon can continue to further insert the stimulating assembly 518 into the cochlea with a lessened risk of the carrier member 552 puncturing fine tissues of the cochlea. It is possible that during the further insertion process the surgeon may simultaneously withdraw the platinum stylet 562 from the lumen 560. Upon withdrawal of the stylet 562, the carrier member 552 is free to adopt its full pre-curved configuration.

As shown in FIG. 4B, disposed in or on the stylet 562 is a first implantable sensor 502A. Additionally, disposed in or on the stiffening sheath 564 is a second implantable sensor 502B. The sensors 502A and 502B are positioned at a distal ends of the stylet 562 and stiffening sheath 564, respectively. The implantable sensors 502A and 502B may be any of the sensors described above. For example, in one embodiment, the implantable sensors 502A and 502B may be force sensors. In another embodiment, the implantable sensors 502A and 502B may be speed or movement sensors. In a further embodiment, the implantable sensor 502A may be a force sensor and the implantable sensor 502B may be a speed sensor.

The locations of implantable sensors 502A and 502B shown in FIG. 4B are merely illustrative and other locations may be used in alternative embodiments. Additionally, although FIG. 4B illustrates the use of two implantable sensor 502, alternative embodiments may include a single implantable sensors for more than two implantable sensors.

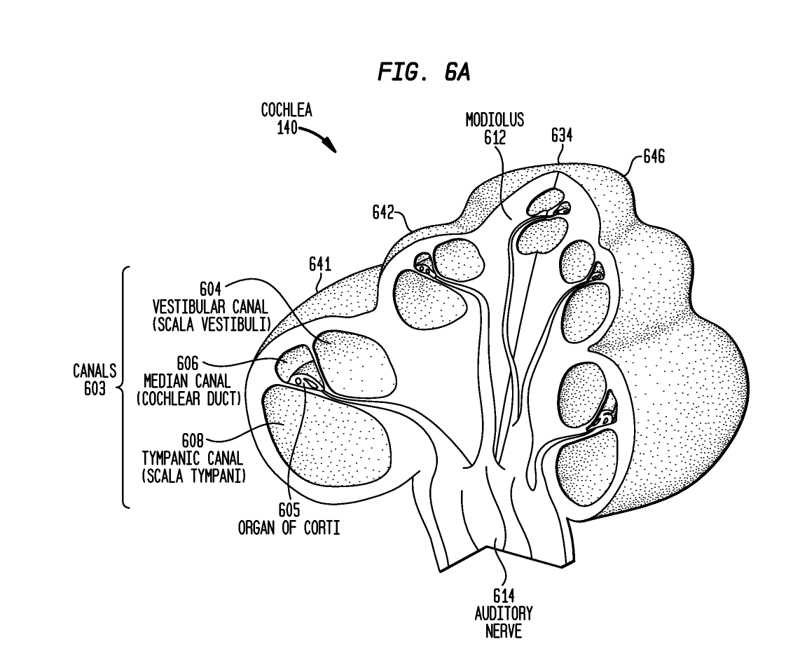

FIG. 5 is perspective view of a device 580 used for inserting a stimulating assembly into a cochlea of a recipient and removing a stiffening element of the stimulating assembly during insertion. The device comprises a housing 581 which extends from a proximal end 582 to a distal end 583. The distal end 583 is adapted to receive a portion of a stimulating assembly 590.

The device 580 further comprises a positioning member (not shown) which extends through the housing 581. The positioning member engages the stiffening element of the stimulating assembly 590 and is actuated via finger rests 592, thereby providing a surgeon with the ability to withdraw the stiffening element from the stimulating assembly 590 during implantation.

As shown in FIG. 5, disposed in or on the device 580 is a sensor 522. Since the device 580 may be used externally to the recipient, the sensor 522 may not be an implantable sensor (i.e., the sensor 522 is need not have a configuration such that it will operate when it is inserted in the recipient). However, the sensor 522 may be measure the same attributed as any of the sensors described above. For example, in one embodiment, the sensor 522 may be a force sensor. In another embodiment, the sensor 522 may be a speed or movement sensor.

The location of sensor 552 shown is merely illustrative and other locations may be used in alternative embodiments. Additionally, although FIG. 5 illustrates the use of a single sensor 552, alternative embodiments may include a plurality of sensors.

FIGS. 3, 4, and 5 illustrate embodiments in which sensors are disposed in or on various tools used in conjunction with an implantable medical device, namely a cochlear implant. It is to be appreciated that further embodiments may include other types of tools with one or more sensors.

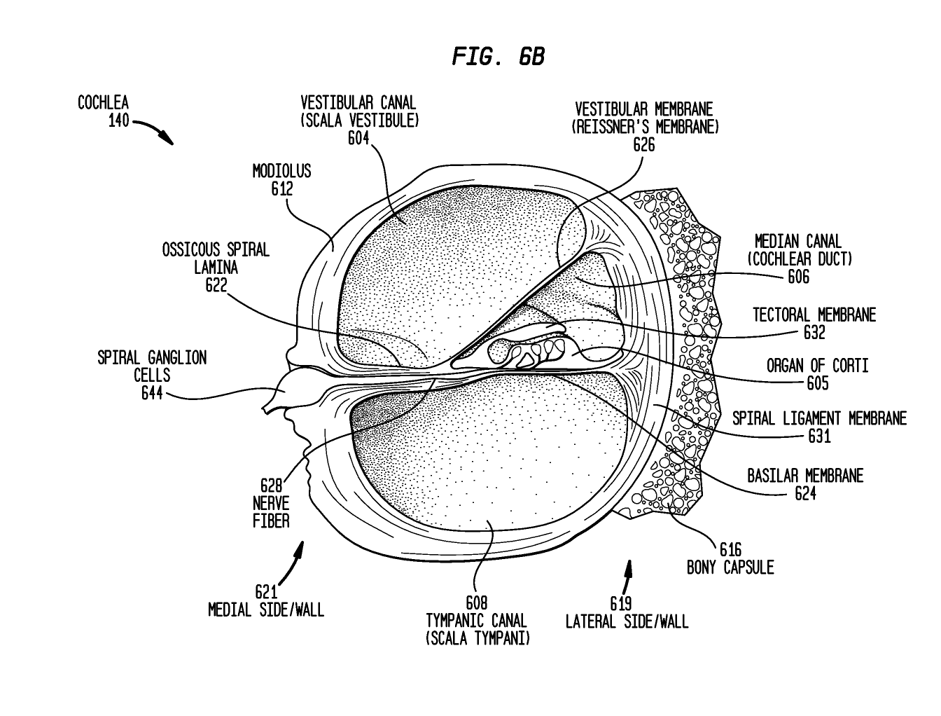

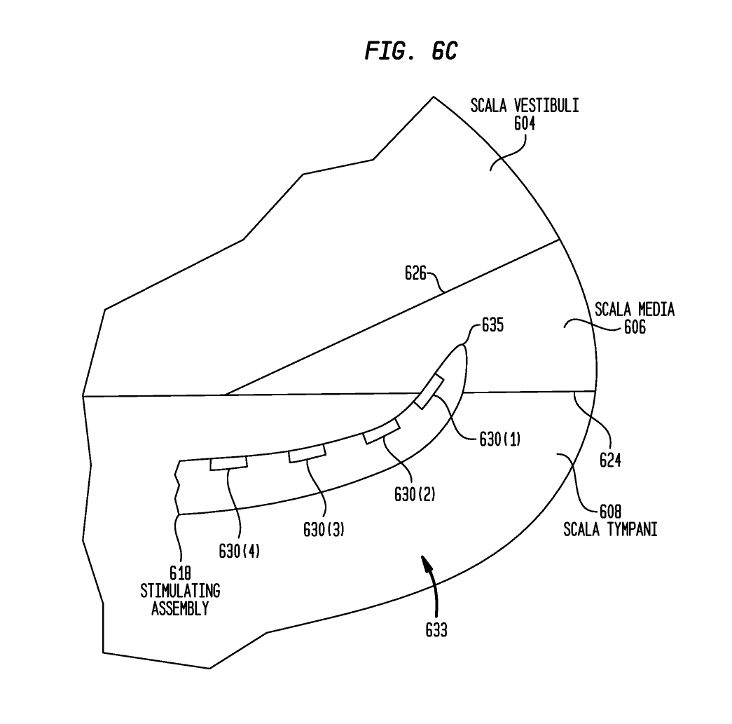

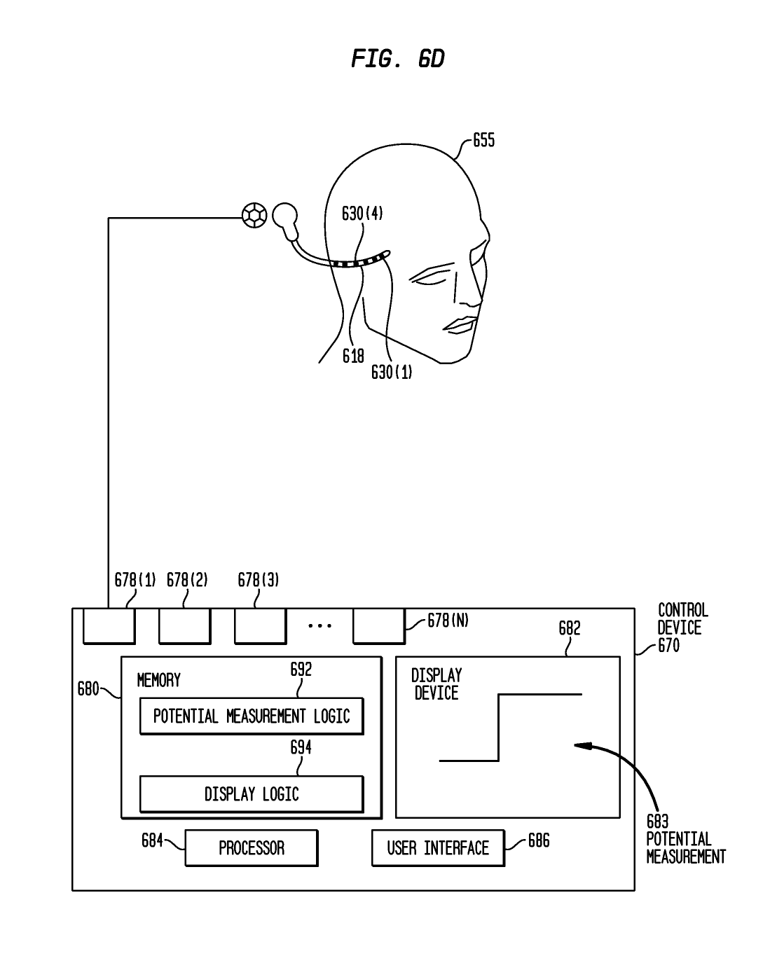

As noted above, implantable sensors in accordance with embodiments presented herein may take a number of different forms to, for example, monitor insertion attributes. For example, in embodiments described above, an implantable sensor may be disposed in the stimulating assembly to detect when the stimulating assembly makes contact with the wall of the cochlea. FIGS. 6C and 6D illustrates one specific such arrangement where a sensor is configured to detect when the stimulating assembly dislocates and penetrates a recipient's basilar membrane during insertion (i.e., stimulating assembly dislocation is a monitored insertion attribute). It is to be appreciated that FIGS. 6C and 6D are merely illustrative and do not necessarily to a how, in practice, a stimulating assembly may be implanted in a cochlea.

Before describing the embodiment of FIGS. 6C and 6D, a brief overview of the relevant aspects of a cochlea are described first with reference to FIGS. 6A and 6B. More specifically, FIG. 6A is a partially cut-away perspective view of the cochlea that illustrates the canals and nerve fibers of the cochlea. FIG. 6B is a cross-sectional view of one turn of the canals of the cochlea. To facilitate understanding, the following description will reference the cochlea illustrated in FIGS. 2A and 2B as cochlea 140, which was introduced above with reference to FIG. 1.

Referring to FIG. 6A, cochlea 140 is a conical spiral structure comprising three parallel fluid-filled canals or ducts, collectively and generally referred to herein as canals 603. Canals 603 comprise the tympanic canal 608, also referred to as the scala tympani 608, the vestibular canal 604, also referred to as the scala vestibuli 604, and the median canal 606, also referred to as the scala media 606. Cochlea 140 spirals about modiolus 612 several times and terminates at cochlea apex 634.

Referring now to FIG. 6B, separating canals 603 of cochlear 140 are various membranes and other tissue. The Ossicous spiral lamina 622 projects from modiolus 612 to separate scala vestibuli 604 from scala tympani 608. Toward lateral side 619 of scala tympani 608, a basilar membrane 624 separates scala tympani 608 from scala media 606. Similarly, toward lateral side 619 of scala vestibuli 604, a vestibular membrane 626, also referred to as the Reissner's membrane 626, separates scala vestibuli 604 from scala media 606.

Portions of cochlea 140 are encased in a bony capsule 616. Bony capsule 616 resides on lateral side 619 (the right side as illustrated in FIG. 6B), of cochlea 140. Spiral ganglion cells 644 reside on the opposing medial side 621 (the left side as illustrated in FIG. 6B) of cochlea 140. A spiral ligament membrane 631 is located between lateral side 619 of spiral tympani 608 and bony capsule 616, and between lateral side 619 of scala media 606 and bony capsule 616. Spiral ligament 631 also typically extends around at least a portion of lateral side 619 of scala vestibuli 604.

In normal hearing, sound entering auricle 110 (FIG. 1) causes pressure changes in cochlea 140 that travel through the fluid-filled scala tympani 608 and the scala vestibular 604. The organ of Corti 605 is situated on basilar membrane 624 in scala media 606. The organ of Corti 605 comprises rows of 16,000-20,000 hair cells (not shown) which protrude from its surface. Above the hair cells is the tectoral membrane 632 which moves in response to pressure variations in the fluid-filled scala tympani 608 and the scala vestibular 604. Small relative movements of the layers of membrane 632 are sufficient to cause the hair cells in the endolymph to move thereby causing the creation of a voltage pulse or action potential which travels along the associated nerve fiber 628. Nerve fibers 628, embedded within spiral lamina 622, connect the hair cells with the spiral ganglion cells 644 which form auditory nerve 614. Auditory nerve 614 relays the impulses to the auditory areas of the brain (not shown) for processing.

The fluid in the scala tympani 608 and the scala vestibular 604 is referred to as perilymph, while the fluid in the scala media 606 (which surrounds the organ of Corti 610) is referred to as endolymph. The perilymph has different properties than that of the endolymph. For example, the perilymph in the scala tympani 608 may have a potential of approximately zero (0) millivolts (mV), while the endolymph in the scala media 606 may have a potential of approximately eighty (80) mV. The perilymph in the scala vestibule 604 may have a potential of approximately two (2) to five (5) mV. FIGS. 6C and 6D illustrate embodiments configured to take advantage of the potential differences between the various areas of the cochlea to detect, for example, when a stimulating assembly dislocated and perforates the basilar membrane 624. Alternatively, or additionally, the sensor of FIGS. 6C and 6D could measure ionic concentrations to determine the location of the distal end of the stimulating assembly.

FIG. 6C illustrates a stimulating assembly 618 comprising a plurality of stimulating contacts 630. For ease of illustration, only four stimulating contacts 630(1)-630(4) at the distal end 633 of the stimulating assembly 618 are shown in FIG. 6C. During implantation, the stimulating assembly 618 is inserted into the scala tympani 608 of the cochlea 140. In certain circumstances, the tip 635 at the distal end 633 can dislocate and penetrate the basilar membrane 624. FIG. 6C illustrates such a circumstance where the tip 635 has penetrated and passed through the basilar membrane 624.

If, after the initial dislocation of the tip 635, the surgeon continues to insert the stimulating assembly 618, the stimulating could potential transverse the scala media 606 and penetrate the vestibular membrane 626 and pass into the scala vestibuli 604. This may lead to a decrease in potential sound perception performance available to the cochlear implant recipient.

In general, surgeons rely upon touch/feel and their previous experiences to determine if the tip 635 of stimulating assembly has dislocated. In order to improve insertion and prevent significant trauma to the cochlea 140 due to continued insertion after tip dislocation, the arrangement of FIGS. 6C and 6D provides the surgeon with real-time feedback when the tip 635 of the stimulating assembly penetrates the basilar membrane. More specifically, in these embodiments, the stimulating contacts 630 and/or a reference electrode (not shown) operate as sensors that measure the potential at (adjacent to) the stimulating contacts 630 during insertion. That is, as the stimulating assembly 618 is inserted into the cochlea 140, one or more of the stimulating contacts 630 are used to continually (or periodically) monitor the potential adjacent to the respective contact.

Initially, when the stimulating assembly 618 is positioned in the scala typmani 608, the stimulating contacts 630 may all measure substantially the same potential. However, if dislocation occurs, a potential change (e.g., the potential difference between the perilymph in scala tympani 608 and the endolymph in scala media 606 and/or the potential difference between the perilymph in scala tympani 608 and the basilar membrane 624) may be detected between the most distal stimulating contact 630(1) and the other stimulating contacts 630(2)-630(4). For example, after penetration of the basilar membrane 624, the most distal stimulating contact 630(1) may measure a potential of 80 mV (corresponding to the potential of the endolymph) while the other stimulating contacts 630(2)-630(4) measure a potential of 0 mV (corresponding to the perilymph of the scala tympani 608). If such a potential difference is noted, feedback may be provided to the surgeon indicating that dislocation of the stimulating assembly 618 has occurred. In certain embodiments, a voltage sensor is placed in between the tip electrode and the other more basally oriented electrodes and two voltage sensors are not utilized (e.g., a relatively small sensing electrode at the tip and the other contacts are used as reference contacts.

Alternatively, if the most distal contact 630(1) is in contact with the basilar membrane 624, the most distal stimulating contact 630(1) may measure a potential of −60 mV (corresponding to the potential of the basilar membrane 624) while the other stimulating contacts 630(2)-630(4) measure a potential of 0 mV (corresponding to the perilymph of the scala tympani 608). If such a potential difference is noted, feedback may be provided to the surgeon indicating that the distal end 633 of the stimulating assembly 618 is in contact with the basilar membrane 624 (e.g., feedback indicating that dislocation is likely or has occurred).

In general, changes in ionic environment will occur and would be measureable as impedance changes between the stimulating contacts 630(1)-630(4). The cochlear implant system may be configured to perform multiple real-time measurements substantially simultaneously and use these multiple measurements to deduce the actual moment of penetration. For example, assuming that the penetration occurs with tip 635, a voltage jump between the most distal stimulating contacts shortly followed by a lowering in impedance in between these contacts due to the higher conductive (high ionic) environment may indicate tip dislocation. The cochlear implant system could also be configured to detect dislocation based on other circumstances (e.g., the spiral ligament with its ion pumps may be a potential source that is possibly observable when a penetrated electrode gets close to the spiral ligament).

The perilymph and the endolymph include different ionic concentrations. For example, the perilymph may contain approximately the following ionic concentrations: Na at approximately 140 mM, K at approximately 4-5 mM, Cl at approximately 110 mM, and Ca at approximately 1.2 mM. The endolymph may contain approximately the following ionic concentrations: Na at approximately 1 mM, K at approximately 150 mM, Cl at approximately 130 mM, and Ca at approximately 0.2 mM. In certain embodiments, an ionic concentration sensor could measure the ionic concentrations adjacent thereto for use in detecting dislocation of the distal end of the stimulation assembly. More specifically, and detected differences in ionic concentrations may be detected and used to determine the location of the distal end of the stimulating assembly.

FIG. 6D illustrates a control device 670 that may used to display an indication of measured potential changes based on signals received from stimulating contacts 630. Control device 670 is a computing device that comprises a plurality of interfaces/ports 678(1)-678(N), a memory 680, a display device (e.g., screen) 682, a processor 684, and a user interface 686.

The interfaces 678(1)-678(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 6D, interface 678(1) is connected to an external coil 625 that receives signals from a cochlear implant 600 that includes stimulating assembly 618. Interface 678(1) may be alternatively connected to an external device that is communication with the external coil or cochlear implant 600. Interface 678(1) may be configured to receive the signals via a wired or wireless connection (e.g., telemetry, Bluetooth, etc.).

The memory 680 includes potential measurement logic 692 and imaging logic 694. In certain embodiments, the potential measurement logic 692 may be executed to monitor potentials at the stimulating contacts 630 and/or process signals from the stimulating contacts 630. The potential measurement logic 692 may execute an algorithm that, for example, uses measurements from multiple stimulating contacts and/or a reference electrode, identifies and omits “false” positive or negative measurements, etc.

The display logic 694 may use data generated by the potential measurement logic 692 to display a potential measurement 683. In certain examples, the display device 682 may display an indication that a large potential difference has been detected between the distal stimulating contact 630(1) and the other stimulating contacts. Additionally, the display device 682 may display a notification or indication that dislocation or contact with the basilar membrane 624 has occurred. The control device 670 may also or alternatively generate other types of feedback to alert the surgeon that that dislocation or contact with the basilar membrane 624 has occurred.

Memory 680 may comprise read only memory (ROM), random access memory (RAM), magnetic disk storage media devices, optical storage media devices, flash memory devices, electrical, optical, or other physical/tangible memory storage devices. The processor 684 is, for example, a microprocessor or microcontroller that executes instructions for the potential measurement logic 692 and the display logic 694. Thus, in general, the memory 680 may comprise one or more tangible (non-transitory) computer readable storage media (e.g., a memory device) encoded with software comprising computer executable instructions and when the software is executed (by the processor 684) it is operable to perform the operations described herein in connection with potential measurement logic 692 and display logic 694.

The embodiments of FIGS. 6C and 6D may provide surgeons with direct and real-time feedback during insertion of the stimulating assembly 618. This may advance their surgical approach and skills, but also prevent cochlea damage that could potentially occur if a dislocated stimulating assembly is advanced into the cochlea.

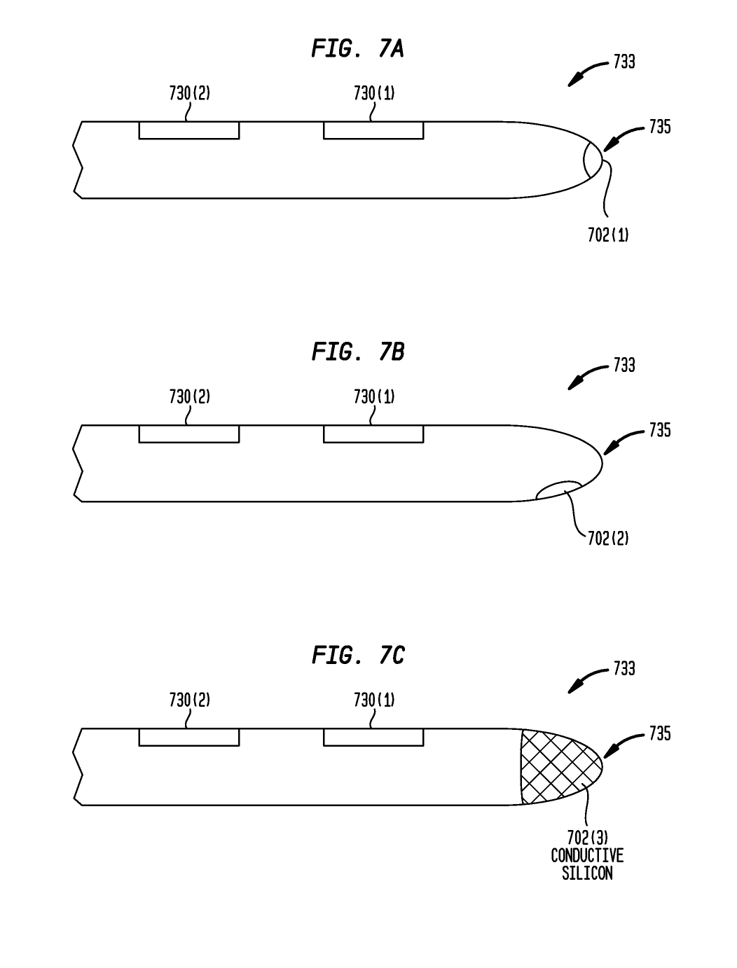

As noted above, an implantable sensor in accordance with certain embodiments may comprise a proximity sensor that is configured to detect when the stimulating assembly is close to a cochlear wall and/or provide an indication of the distance between the stimulating assembly and a cochlea wall (i.e., proximity of the stimulating assembly to a cochlea wall is a monitored insertion attribute). Such proximity sensors may assist with the final placement of the stimulating assembly in the cochlea. Proximity sensors may operate based on electrical, electrochemical or electro-neural properties (e.g., voltage, current, impedance, neural potential, etc.) In certain embodiments, proximity sensors may provide an objective or defined measure of the proximity of the stimulating assembly to a cochlea structure. In other embodiments, the output from a proximity sensor may be used to produce a reconstructed image of the cochlea (or a portion thereof). FIGS. 7A-7C are schematic diagrams illustrating proximity sensors in accordance with embodiments of the present invention that are configured to detect when the distal tip of a stimulating assembly contacts the wall of a recipient's cochlea. In general, the sensors of FIGS. 7A-7C are conductive stimulating assembly tips configured to perform electrical measurements to detect when the tip contacts, or is close proximity to the wall of a recipient's cochlea.

As noted above, a risk associated with cochlear implants is that the stimulating assembly may dislocate and penetrate the recipient's basilar membrane or another cochlea wall. Cochlea penetration causes tissue damage that can have adverse impacts on the recipient's residual hearing as well as the success of the cochlear implant. Currently, there are no technologies available that provide feedback to the surgeon before dislocation and basilar membrane potential occurs. Rather, the most common practice is to perform post-surgical imaging to confirm correct placement of the stimulating assembly. FIGS. 7A-7C illustrate stimulating assembly designs that enable detection of possible stimulating assembly dislocation before the basilar membrane is perforated.

More specifically, referring first to FIG. 7A, a stimulating assembly 718(1) is shown that comprises a plurality of stimulating contacts 730. For ease of illustration, only two stimulating contacts 730(1) and 730(2) at the distal end 733 of the stimulating assembly 718 are shown in FIG. 7A. The stimulating assembly 718(1) includes a tip 735 that is formed from a conductive material (e.g., platinum). As such, the tip 735 is an electrode contact 702(1). The electrode contact 702(1) is configured to operate as an active surface to perform electrical measurements (e.g., impedance, voltage, etc.) that indicate, for example, contact of the tip 735 with the basilar membrane (e.g., potential dislocation, tip foldover, etc.).

FIG. 7B illustrates an alternative stimulating assembly 718(2) comprising the two stimulating contacts 730(1) and 730(2). The stimulating assembly 718(2) includes an electrode contact 702(2) disposed on the lateral side of the tip 735. The electrode contact 702(2) is formed from a conductive material (e.g., platinum) and is configured to operate as an active surface to perform electrical measurements that indicate, for example, contact of the tip 735 with the basilar membrane (e.g., potential dislocation, tip foldover, etc.).

FIG. 7C illustrates another stimulating assembly 718(3) comprising the two stimulating contacts 730(1) and 730(2). In the example of FIG. 7B, the stimulating assembly 718 includes a conductive silicone tip 702(3) (e.g., silicone infused with conductive material). The conductive silicone tip 702(3) is configured to operate as an active surface to perform electrical measurements that indicate, for example, contact of the tip 735 with the basilar membrane (e.g., potential dislocation, tip foldover, etc.).

The stimulating assemblies 718(A)-718(C) may be used with a control device, such as the control device 618 of FIG. 6D, that receives electrical measurements from the electrical sensing elements. The control device may be configured to execute algorithms that determine if dislocation, tip foldover, etc. has or is likely to occur and to provide a surgeon with real-time feedback.

In addition, or alternatively, the stimulating contacts may be configured to perform electrical measurements in addition to stimulation. That is, additional sensing electrode contacts may not be necessary as the stimulation contacts can operate to perform electrical measurements.

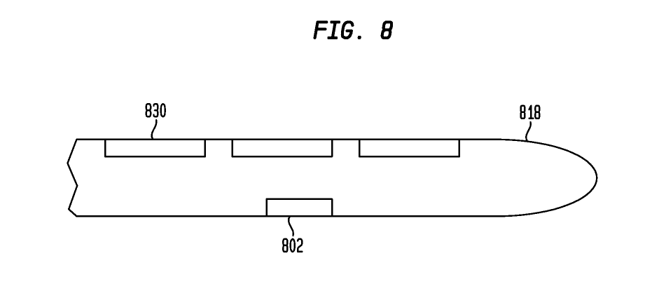

FIG. 8 illustrates a stimulating assembly 818 having another implantable sensor in accordance with embodiments of the present invention. In this embodiment, the stimulating assembly 818 comprises a plurality of stimulating contacts 830 and at least one low impedance electrode contact 802. The low impedance electrode contact 802 may be formed from, for example, platinum oxide or carbon. The low impedance electrode contact 802 operates as a sensor configured to record slow afterpotentials (i.e., potentials present in the cochlear after presentation of a train of electrical stimulation pulses via one or more stimulating contacts 830). That is, the low impedance electrode contact 802 can be used to record slow changes in local potential during stimulation. Additionally, if charge is injected into the cochlea, this can be detected and shorting with the low impedance electrode 802 could be used to overcome the unwanted and potentially traumatic changes in potential. Alternatively, small changes in local potential could be “thropic” to the auditory neurons to cause the activation of spiral ganglion neurons by electric pulses. The low impedance electrode 802 could help to titrate the charge to keep up the local potential within the “throphic” limits. The injection of charge into the cochlea may change the local potential (DC) so as to improve the effectiveness of stimulation pulses.

In certain embodiments, the afterpotentials recorded via the low impedance electrode contact 802 could be stored and subsequently used to determine an optimal rate of stimulation for delivery to the stimulating contacts 830. In particular, the afterpotentials may correspond to neural activity in manner that enables use of the recorded afterpotentials to predict rate effectiveness.

As noted above, an implantable sensor is able to gather information about some parameter (e.g., physical, electrical, chemical, and biological) and output a signal. Embodiments of the present invention are configured to use of the signal(s) provided by implantable sensors in a number of different manners.

In certain embodiments, an implantable sensor may be configured to provide an alert to feedback to a surgeon, the recipient, caregiver, clinician, manufacturer, etc. (collectively and generally referred to as “users”). For example, the feedback may be audible (e.g., an audible warning may be generated when the tip of the stimulating assembly becomes stuck or has begun to perforate the basilar membrane), visual (e.g., display of capture images, reconstructed images, etc.), haptic/tactile (e.g., vibrations, buzzing, etc.). It is also to be appreciated that different types of feedback may be used in combination with one another (i.e., a visual presentation on a display screen along with an audible warning).

Alternatively, or additionally to an alert or feedback, an insertion tool could directly limit the rate of insertion, such as in response to an alarm notification being received. The insertion tool could alternatively control the insertion speed by utilizing the information from the sensor to ensure that the speed is controlled so that the pressure never reaches a pre-determined value. Furthermore, when a second pre-determined pressure is sensed, the insertion tool could pause or reduced in speed, thereby allowing the pressure to decrease to a safe level prior to continuing forward insertion. The continued insertion could then be performed at a lower speed than previously used. The insertion tool that controls the insertion speed could be hand held, fully automated (e.g., floor/ceiling mounted), or a combination of hand held and fully automated.

Furthermore, the signals from an implantable sensor may be used to trigger a variety of actions. The triggered actions may depend, for example, on the type of sensor, the sensed parameters, etc. For example, in certain embodiments the implantable sensor may trigger the release of a drug or drugs into the cochlea or an alert on the remote assistant to contact medical practitioner (e.g., a clinician or audiologist) with the purpose of an action that should be performed or of a potential threat that should be mitigated. Alternatively, the signals from the implantable sensor may be logged (e.g., stored) within the cochlear implant at the implantable component and/or the external component. These logged sensor results may be used to inform an audiologist or other user of an action that should be performed or of a potential threat (not real time, but delayed). Alternatively, these logged results may be provided to the device manufacturer for use in, for example, post market surveillance, quality control, system design, etc.

In further embodiments, the signals from the implantable sensor may cause a shut down of the cochlear implant or a cessation of electrical stimulation. This may be useful in circumstances in which a danger to the recipient is detected. In other embodiments, the signals from the implantable sensor may be used to change or adjust the operation of the device. For example, the signals may be used to change the stimulation strategy implemented (e.g., rate, current level, disable some certain electrical contacts, switch between monopolar and bi-polar stimulation modes, etc.)

In alternative embodiments, the signals form an implantable sensor may be used to assist in robotic insertion of a stimulating assembly. A robotic insertion device may use the signals to, for example, halt the procedure, change speed, change direction (e.g., angle or reverse), disengage robot from device (i.e., when the stimulating assembly is properly positioned), etc.

It is appreciated that implantable sensors may sense or monitor parameters in different manners. For example, certain sensors may continuously monitor parameters, while other sensors may sense parameters at discrete times (e.g., periodically, randomly, etc.) Implantable sensors may begin operation immediately upon insertion into a recipient, begin operation after a predetermined period of time, or begin operation after certain event (e.g., begin monitoring one week after a drug has been released).

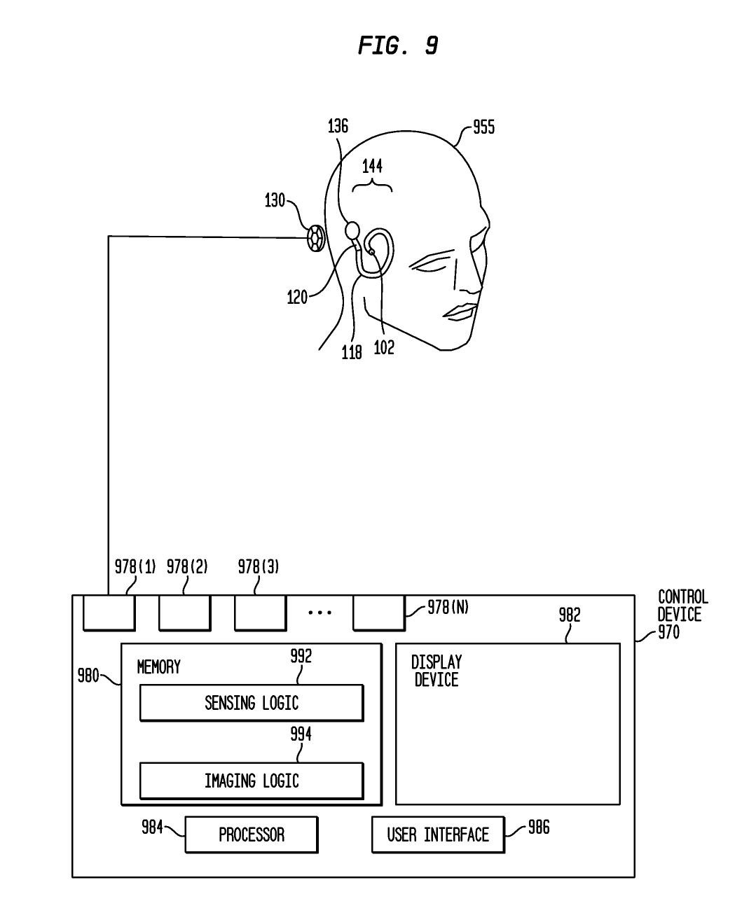

As noted above, in accordance with certain embodiments, an implantable sensor in accordance with embodiments presented herein may be a device configured to produce an image of, or assist in production of a reconstructed image of, the recipient's cochlea. FIG. 9 is a schematic diagram illustrating the insertion of a stimulating assembly having such an implantable sensor into a recipient. For ease of reference, the embodiments of FIG. 6 will be described with reference to the components of cochlear implant 100 of FIGS. 1, 2A, and 2B during insertion into a recipient 955.

FIG. 9 illustrates a control device 970 that is used to display an image (e.g., either a captured or reconstructed image) based on signals received from an implantable sensor 102. Control device 970 is a computing device that comprises a plurality of interfaces/ports 978(1)-978(N), a memory 980, a display device (e.g., screen) 982, a processor 984, and a user interface 986.

The interfaces 978(1)-978(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 9, 978(1) is connected to external coil 130 and/or an external device in communication with the external coil. Interface 978(1) may be configured to receive the voltage signals via a wired or wireless connection (e.g., telemetry, Bluetooth, etc.).

The memory 980 includes sensing logic 992 and imaging logic 994. In certain embodiments, the sensing logic 992 may be executed to sample implantable sensor 102 and/or process signals from the implantable sensor 102. The imaging logic 994 may use data generated by the sensing logic 992 to display a captured or reconstructed image of the recipient's cochlea, as well as a representation of the stimulating assembly 118, at display device 382.

Memory 980 may comprise read only memory (ROM), random access memory (RAM), magnetic disk storage media devices, optical storage media devices, flash memory devices, electrical, optical, or other physical/tangible memory storage devices. The processor 984 is, for example, a microprocessor or microcontroller that executes instructions for the sensing logic 992 and an imaging logic 994. Thus, in general, the memory 980 may comprise one or more tangible (non-transitory) computer readable storage media (e.g., a memory device) encoded with software comprising computer executable instructions and when the software is executed (by the processor 984) it is operable to perform the operations described herein in connection with sensing logic 992 and an imaging logic 994.

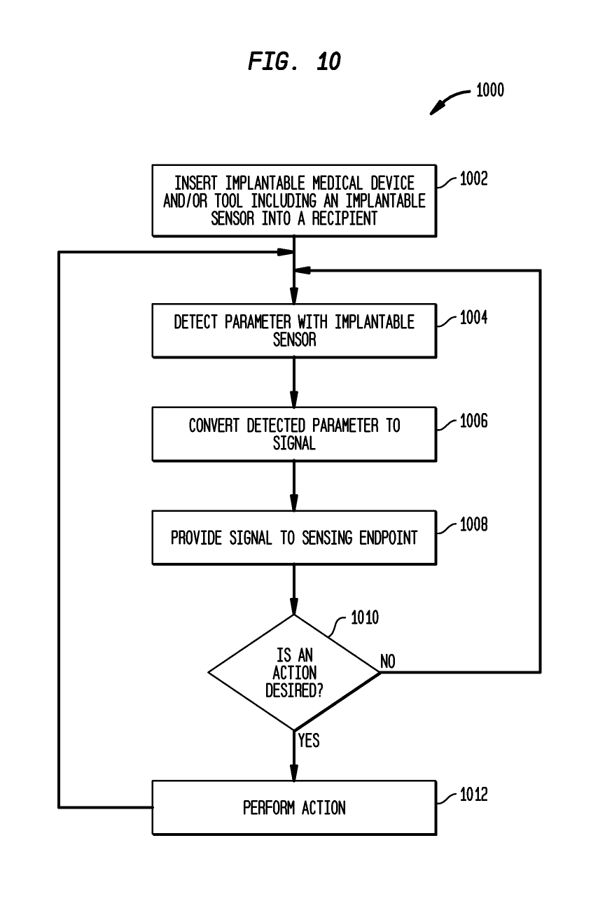

FIG. 10 is a flowchart of a method 1000 in accordance with embodiments presented herein. Method 1000 begins at 1002 where an implantable medical device and/or tool that include an implantable sensor are inserted into a recipient. At 1004, a parameter (e.g., physical, electrical, chemical, biological parameter) is detected with the implantable sensor. In certain embodiments, the parameter relates to a monitored insertion attribute. At 1006, the detected parameter is converted into a signal and at 1008 the signal is provided to a sensing endpoint (e.g., computing device, a portion of the cochlear implant, etc.). At 1010, a determination is made as to whether an action, such as providing feedback, is desired. If not, the method 1000 may return to 1004 for additional parameter detection. If an action is desired, method 1000 proceeds to 1012 where the action is performed. After the action is completed, the method 1000 may return to 1004 for additional parameter detection.

Embodiments have been generally been described herein with reference to implantable sensors used in/on, or in conjunction with, cochlear implants. However, in accordance with embodiments presented herein, implantable sensors may be used with other implantable medical devices having a wide variety of corresponding implantable components that may be partially or fully implanted into a recipient. For example, in accordance with embodiments presented herein, hearing prostheses (e.g., auditory brain stimulators, bone conduction devices, mechanical stimulators, middle ear implants, cochlear implants, etc.), implantable pacemakers, defibrillators, functional electrical stimulation devices, catheters, etc., may include one or more implantable sensors. Additionally, in accordance with embodiments presented herein, implantable sensors may be in/on different types of instruments that may be used in with these implantable medical devices (e.g., different types of tools used during implantation, explantation, etc. of an implantable medical device).

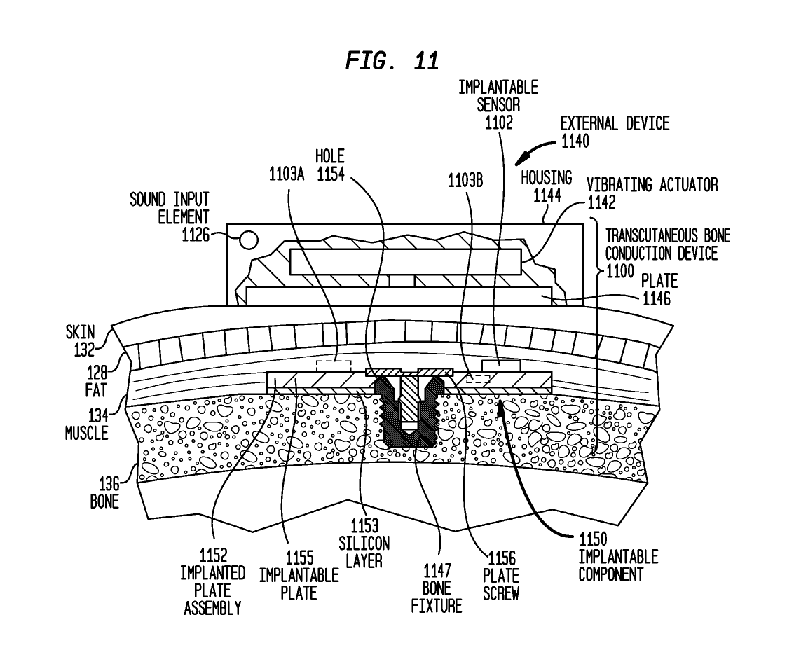

FIG. 11 depicts an exemplary embodiment of a transcutaneous bone conduction device 1100 that includes an external device 1140 and an implantable component 1150. The implantable component 1150 includes an implantable sensor 1102.

The transcutaneous bone conduction device 1100 of FIG. 11 is a passive transcutaneous bone conduction device in which a vibrating actuator 1142 is located in the external device 1140. Vibrating actuator 1142 is located in housing 1144 of the external component, and is coupled to plate 1146. Plate 1146 may be in the form of a permanent magnet and/or in another form that generates and/or is reactive to a magnetic field, or otherwise permits the establishment of magnetic attraction between the external device 1140 and the implantable component 1150 sufficient to hold the external device 1140 against the skin 132 of the recipient.

In an exemplary embodiment, the vibrating actuator 1142 is a device that converts electrical signals into vibration. In operation, sound input element 1126 converts sound into electrical signals that are provided to vibrating actuator 1142, or to a sound processor (not shown) that processes the electrical signals, and then provides those processed signals to vibrating actuator 1142. The vibrating actuator 1142 converts the electrical signals (processed or unprocessed) into vibrations. Because vibrating actuator 1142 is mechanically coupled to plate 1146, the vibrations are transferred from the vibrating actuator 1142 to plate 1146.

Implanted plate assembly 1152 is part of the implantable component 1150, and is made of a ferromagnetic or ferromagnetic material that may be in the form of a permanent magnet, that generates and/or is reactive to a magnetic field, or otherwise permits the establishment of a magnetic attraction between the external device 1140 and the implantable component 1150 sufficient to hold the external device 1140 against the skin of the recipient. Accordingly, vibrations produced by the vibrating actuator 1142 of the external device 1140 are transferred from plate 1146 across the skin to plate 1155 of plate assembly 1152. This may be accomplished as a result of mechanical conduction of the vibrations through the skin, resulting from the external device 1140 being in direct contact with the skin and/or from the magnetic field between the two plates. These vibrations are transferred without penetrating the skin with a solid object such as an abutment as detailed above with respect to a percutaneous bone conduction device.

As may be seen, the implanted plate assembly 1152 is substantially rigidly attached to bone fixture 1147 in this embodiment. In this regard, implantable plate assembly 1152 includes a through-hole 1154 that is contoured to the outer contours of the bone fixture 1147. Plate screw 1156 is used to secure plate assembly 1152 to bone fixture 1147. As can be seen in FIG. 11, the head of the plate screw 1156 is larger than the hole through the implantable plate assembly 1152, and thus the plate screw 1156 positively retains the implantable plate assembly 1152 to the bone fixture 1147.

As shown in FIG. 11, disposed in or on the implantable component 1150 is an implantable sensor 1102. The implantable sensor 1102 may be any of the sensors described above. Although FIG. 11 illustrates the use of a single implantable sensor 1102, alternative embodiments may include a plurality of sensors.

The location of implantable sensor 1102 shown in FIG. 11 is also merely illustrative and other locations may be used in alternative embodiments. For example, dashed boxes 1103A and 1103B illustrate other potential locations for an implantable sensor in accordance with embodiments presented herein.

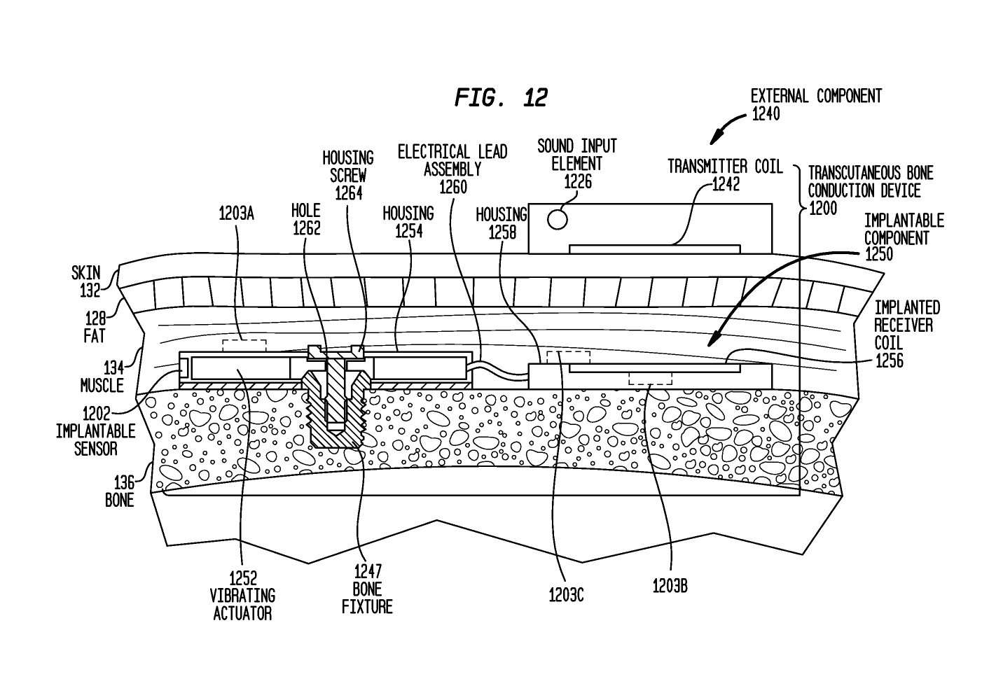

FIG. 12 depicts an exemplary embodiment of a transcutaneous bone conduction device 1200 according to another embodiment of the present invention. Bone conduction device 1200 comprises an external device 1240 and an implantable component 1250 that includes an implantable sensor 1202. The transcutaneous bone conduction device 1200 of FIG. 12 is an active transcutaneous bone conduction device in that the vibrating actuator 1252 is located in the implantable component 1250. Specifically, a vibratory element in the form of vibrating actuator 1252 is located in housing 1258 of the implantable component 1250. In an exemplary embodiment, much like the vibrating actuator 842 described above with respect to transcutaneous bone conduction device, the vibrating actuator 1252 is a device that converts electrical signals into vibration.

External component 1290 includes a sound input element 1226 that converts sound into electrical signals and provides these electrical signals to vibrating actuator 1252, or to a sound processor (not shown) that processes the electrical signals, and then provides those processed signals to the implantable component 1250 through the skin of the recipient via a magnetic inductance link. In this regard, a transmitter coil 1292 of the external component 1240 transmits these signals to implanted receiver coil 1256 located in housing 1258 of the implantable component 1250. Components (not shown) in the housing 1258, such as, for example, a signal generator or an implanted sound processor, then generate electrical signals to be delivered to vibrating actuator 1252 via electrical lead assembly 1260. The vibrating actuator 1252 converts the electrical signals into vibrations. The vibrating actuator 1252 is mechanically coupled to the housing 1258. Housing 1258 and vibrating actuator 1252 collectively form a vibrating element. The housing 1258 is substantially rigidly attached to a bone fixture 1247. In this regard, housing 1258 includes through hole 1262 that is contoured to the outer contours of the bone fixture 1247. Housing screw 1264 is used to secure housing 1258 to bone fixture 1247.

As shown in FIG. 12, disposed in or on the implantable component 1250 is an implantable sensor 1202. The implantable sensor 1202 may be any of the sensors described above. Although FIG. 12 illustrates the use of a single implantable sensor 1202, alternative embodiments may include a plurality of sensors.

The location of implantable sensor 1202 shown in FIG. 12 is also merely illustrative and other locations may be used in alternative embodiments. For example, dashed boxes 1203A, 1203B, and 1203C illustrate other potential locations for an implantable sensor in accordance with embodiments presented herein.

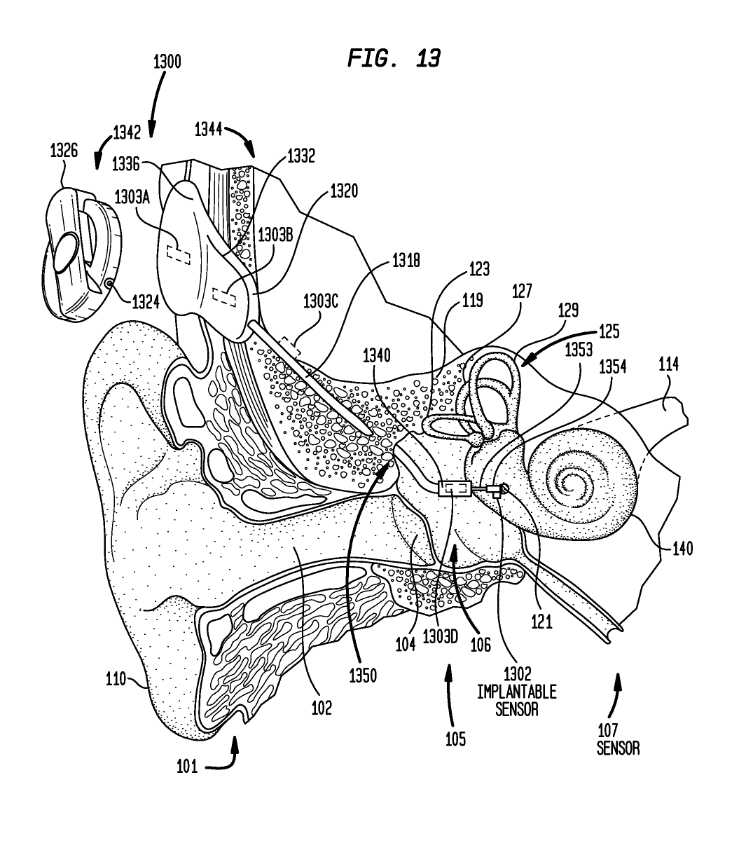

FIG. 13 is a perspective view of a direct acoustic stimulator 1300, comprising an external component 1342 which is directly or indirectly attached to the body of the recipient, and an internal component 1344 that includes an implantable sensor 1302. External component 1342 typically comprises one or more sound input elements 1324, sound processing unit 1326, a power source (not shown), and an external transmitter unit (also not shown). Internal component 1344 comprises internal receiver unit 1332, stimulator unit 1320, stimulation arrangement 1350, and an implantable sensor 1302.

Stimulation arrangement 1350 is implanted in middle ear 105. For ease of illustration, ossicles 106 have been omitted from FIG. 13. However, it should be appreciated that stimulation arrangement 1350 can be implanted without disturbing ossicles 106. Stimulation arrangement 1350 comprises actuator 1340, stapes prosthesis 1354 and coupling element 1353 connecting the actuator to the stapes prosthesis. In this example, stimulation arrangement 1350 is implanted and/or configured such that a portion of stapes prosthesis 1354 abuts round window 121. It should be appreciated that stimulation arrangement 1350 may alternatively be implanted such that stapes prosthesis 1354 abuts an opening in horizontal semicircular canal 123, in posterior semicircular canal 127 or in superior semicircular canal 129.

A sound signal is received by one or more microphones 1324, processed by sound processing unit 1326, and transmitted as encoded data signals to internal receiver 1332. Based on these received signals, stimulator unit 1320 generates drive signals that cause actuation of actuator 1340. This actuation is transferred to stapes prosthesis 1354 such that a wave of fluid motion is generated in the perilymph in scala tympani. Such fluid motion, in turn, activates the hair cells of the organ of Corti. Activation of the hair cells causes appropriate nerve impulses to be generated and transferred through the spiral ganglion cells (not shown) and auditory nerve 114 to the brain (also not shown) where they are perceived as sound.

It should be noted that FIG. 13 is but one example of a direct acoustic stimulator, and in other arrangements, other types of direct acoustic stimulator can be implemented. Further, although FIG. 13 provides an illustrative example of a direct acoustic stimulator system, in other configurations, a middle ear mechanical stimulation device can be configured in a similar manner, with the exception that instead of the actuator 1340 being coupled to the inner ear of the recipient, the actuator is coupled to the middle ear of the recipient. For example, the actuator may stimulate the middle ear by direct mechanical coupling via a coupling element attached to the ossicles.

As shown in FIG. 13, disposed in or on the implantable component 1344 is an implantable sensor 1302. The implantable sensor 1302 may be any of the sensors described above. Although FIG. 13 illustrates the use of a single implantable sensor 1302, alternative embodiments may include a plurality of sensors.

The location of implantable sensor 1302 shown in FIG. 13 is also merely illustrative and other locations may be used in alternative embodiments. For example, dashed boxes 1303A-1303D illustrate other potential locations for an implantable sensor in accordance with embodiments presented herein.

A number of different embodiments have been described herein. It is to be appreciated that these embodiments are not mutually exclusive, but rather may be used in various combinations. Additionally, the invention described and claimed herein is not to be limited in scope by the specific embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A hearing prosthesis, comprising:
an implantable stimulating assembly configured to be implanted in a recipient's cochlea; and
at least one implantable sensor disposed in the implantable stimulating assembly configured to monitor an insertion attribute.

2. The hearing prosthesis of claim 1, wherein the at least one implantable sensor comprises a pressure sensor configured to monitor the fluid pressure within the cochlea during insertion of the implantable stimulating assembly.

3. The hearing prosthesis of claim 2, wherein the pressure sensor is disposed at a distal end of the implantable stimulating assembly.

4. The hearing prosthesis of claim 2, wherein the pressure sensor is a micro silicone pressure sensor.

5. The hearing prosthesis of claim 2, further comprising:
an external device configured to generate feedback to a user when the fluid pressure within the cochlea during insertion of the implantable stimulating assembly reaches a first threshold level.

6. The hearing prosthesis of claim 5, wherein the external device is configured to generate feedback to a user when the fluid pressure within the cochlea during insertion of the implantable stimulating assembly reaches a second threshold level.

7. The hearing prosthesis of claim 6, wherein the first threshold level is set below a fluid pressure level where damage to the cochlea is likely to occur, and the second threshold level is set above the fluid pressure level where damage is likely to occur.

8. The hearing prosthesis of claim 1, wherein the at least one implantable sensor is an accelerometer configured to monitor the speed at which the implantable stimulating assembly is inserted.

9. The hearing prosthesis of claim 1, wherein the at least one implantable sensor is a pressure sensor disposed at a distal end of the stimulating assembly and configured to detect when the distal end of the implantable stimulating assembly contacts a wall of the cochlea during insertion.

10. The hearing prosthesis of claim 1, wherein the at least one implantable sensor is a force sensor configured to determine when the implantable stimulating assembly impacts a wall of the cochlea during insertion.

11. The hearing prosthesis of claim 1, wherein the at least one implantable assembly is an electrical sensor disposed at the distal tip of the implantable stimulating assembly configured to detect proximity of the distal tip to a wall of the recipient's cochlea.

12. The hearing prosthesis of claim 11, wherein the electrical sensor comprises conductive silicone.

13. A method for implanting a hearing prosthesis in a recipient, comprising:
inserting an implantable stimulating assembly in a cochlea of the recipient; and
during insertion of the implantable stimulating assembly, monitoring an insertion attribute with at least one implantable sensor disposed in the implantable stimulating assembly.

14. The method of claim 13, wherein monitoring an insertion attribute comprises:
monitoring, with a pressure sensor, fluid pressure within the cochlea during insertion of the implantable stimulating assembly.

15. The method of claim 14, wherein the pressure sensor is disposed at a distal end of the implantable stimulating assembly.

16. The method of claim 14, wherein the pressure sensor is a micro silicone pressure sensor.

17. The method of claim 14, further comprising:
generating feedback to a user at an external device when the fluid pressure within the cochlea during insertion of the implantable stimulating assembly reaches a first threshold level.

18. The method of claim 17, further comprising:
generating feedback to the user when the fluid pressure within the cochlea during insertion of the implantable stimulating assembly reaches a second threshold level.

19. The method of claim 18, wherein the first threshold level is set below a fluid pressure level where damage to the cochlea is likely to occur, and the second threshold level is set above the fluid pressure level where damage is likely to occur.

20. The method of claim 13, wherein monitoring an insertion attribute comprises:
monitoring, with an accelerometer, the speed at which the implantable stimulating assembly is inserted.
